(19) United States
(12) Patent Application Publication
(10) Pub. No.: US 2005/0191261 A1
(43) Pub. Date: Sep. 1, 2005

(54) Process for strengthening hair and improving the compatibility of a treatment for waving the hair with another subsequent cosmetic treatment comprising applying derivatives of 2-iminothiolane

(76) Inventors: Philippe Barbarat, Bois-Colombes (FR); Herve Andrean, Paris (FR); Chrystelle Rayee, Aulnay-Sous-Bois (FR)

Correspondence Address: Thomas L. Irving;FINNEGAN, HENDERSON, FARABOW,, GARRETTE & DUNNER, L.L.P., 901 New York Avenue, N.W., Washington, DC, 20001-4413, US

(21) Appl. No.: 11/045,301

(22) Filed: Jan. 31, 2005

Related U.S. Application Data
(60) Provisional application No. 60/559,026, filed on Apr. 5, 2004.

(30) Foreign Application Priority Data
Jan. 29, 2004 (FR) 04 50164

(51) Int. Cl. A61K 7/09

(52) U.S. Cl. 424/70.2

(57) ABSTRACT

A process for strengthening the hair, and a process for improving the compatibility of a treatment for waving the hair with a subsequent different cosmetic treatment are disclosed herein. The processes comprise applying to the hair a composition at neutral pH comprising, in a cosmetically acceptable medium, at least one derivative of 2-iminothiolane of formula (1), and/or the water soluble salts thereof.

This application claims benefit under 35 U.S.C. §119 of U.S. Provisional Application No. 60/559,026, filed Apr. 5, 2004, and of French Application No. 04/50164, filed Jan. 29, 2004, both of which are incorporated herein by reference.

The present disclosure relates to processes for the treatment of hair, using derivatives of 2-iminothiolane (2-IT) at neutral pH.

More specifically, the present disclosure relates to a process for strengthening the hair, which can also improve the compatibility of a treatment for waving the hair with a different subsequent cosmetic treatment, this process comprising applying to the hair a composition at neutral pH comprising at least one derivative of 2-iminothiolane.

The derivatives of 2-iminothiolane are known in the art, for example, as coupling agents. Thus, for example, U.S. Pat. No. 5,395,946 describes the use of the 2-iminothiolane derivatives as a coupling agent in the reaction between a protein and a radionuclide. In the publication Textile Res. J. 72,285-289 (2002), it is shown that treatment of wool with 2-iminothiolane at neutral pH promotes the disappearance of undesirable creases.

However, no document describes the use of 2-iminothiolane derivatives for strengthening the hair or for improving the compatibility of a treatment for waving the hair with a different subsequent cosmetic treatment.

With respect to the waving of the hair, for instance, permanent deformation, the procedure generally used comprises, in a first step, opening the disulfide (—S—S—) bridges of the keratin (cystine) by applying to the hair previously placed under tension (via curlers and the like) a reducing composition (reduction step) then, for example, after having rinsed the hair thus treated, of reconstituting in a second step the disulfide bridges by applying to the hair still under tension an oxidizing composition (oxidation step, also called the fixation step) so as to finally give to the hair the desired form.

The reducing compositions that can be used for the implementation of the first step of a conventional process for permanent deformation of the hair (two-step process) may comprise thiol-containing compounds, such as thioglycolic acid or cysteine, or compounds generating hydroxide ions, such as sodium hydroxide or guanidine hydroxide (optionally generated in situ) for example. However, this procedure does not always give completely satisfactory results. For example, if a coloration is applied to hair that is permanently deformed according to the procedure previously described, the color obtained may be different from the color normally obtained with natural hair not treated in such a manner.

It has been suggested that thioglycolic acid be replaced by 2-iminothiolane in order to limit the chemical degradation of the hair. Thus, U.S. Pat. No. 4,263,277 describes the use of a composition with a pH ranging from 9.2 to 9.5, comprising 2-iminothiolane for the permanent deformation of the hair. In the publication J. Appl. Polym. Science 90, 3646-3651 (2003), it is shown that a hair treatment with 2-iminothiolane at a pH equal to 8 or 9 leads can lead to a permanent waving of the fibre.

However, nothing is said concerning the use of 2-iminothiolane derivatives at neutral pH for improving the compatibility of a treatment for waving the hair with a different subsequent cosmetic treatment, for example a treatment for coloring the hair.

Accordingly, the present disclosure provides, on the one hand, a process for strengthening the hair and, on the other hand, a process for improving the compatibility of a treatment for waving the hair with a different subsequent cosmetic treatment.

As used herein, the phrase “different subsequent cosmetic treatment” is understood to mean any treatment other than a treatment for waving the hair, for instance, a treatment for coloring the hair.

Thus, one aspect of the present disclosure is a process for strengthening the hair, comprising applying to the hair a composition of neutral pH comprising, in a cosmetically acceptable medium, at least one compound chosen from 2-iminothiolane derivatives of formula (1) and water-soluble salts thereof: 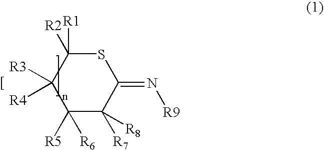
wherein:

R1 to R8, which may be identical or different, are chosen from hydrogen atoms; linear and branched C₁-C₄ hydrocarbon chains, optionally comprising at least one unsaturation, optionally substituted by at least one group chosen from thiol, C₁-C₄ thioalkyl, C₁-C₄ alkylthiol, C₂-C₈ N,N-dialkylamine, C₂-C₄ alkylcarbonylthiol and C₂-C₄ N-alkylcarbonylamine groups; and C₆ aryl groups optionally substituted with at least one entity chosen from halogen atoms, thiol groups, and linear and branched C₁-C₄ alkyl groups,

R9 is chosen from a hydrogen atom and C₁-C₄ linear and branched hydrocarbon chains, and

n is equal to 0 or 1.

The application of compositions comprising derivatives of formula (1) and their water-soluble salts, by increasing the rigidity of the hair, leads to a strengthening of the hair and can makes it possible to increase the volume of the hair.

The strengthening process according to the present disclosure can be implemented independently of any other hair treatment. The process can also be implemented in combination with another hair treatment, i.e. before, during or after the other treatment. The other hair treatment can be any conventional hair treatment, for example, a shampoo, an after-shampoo, a permanent or semi-permanent waving, a straightening or a hairdressing.

Another aspect of the present disclosure is a process for improving the compatibility of a treatment for waving the hair with a subsequent cosmetic hair treatment other than a treatment for waving the hair. The presently disclosed process differs from the known process for waving the hair, the known process comprising a reduction step followed by a fixation step, by substituting the initial reduction step with a treatment step comprising applying to hair a composition of neutral pH comprising, in a cosmetically acceptable medium, at least one compound chosen from 2-iminothiolane derivatives of formula (1) and the water-soluble salts thereof: 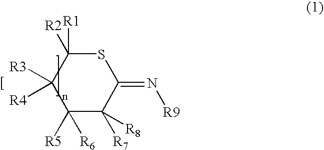
wherein:

R1 to R8, which can be identical or different, are chosen from hydrogen atoms; linear and branched C₁-C₄ hydrocarbon chains, optionally comprising at least one unsaturation, optionally substituted with at least one group chosen from thiol, C₁-C₄ thioalkyl, C₁-C₄ alkylthiol, C₂-C₈ N,N-dialkylamine, C₂-C₄ alkylcarbonylthiol and C₂-C₄ N-alkylcarbonylamine groups; and C₆ aryl groups optionally substituted with at least one entity chosen from halogen atoms, thiol groups and linear and branched C₁-C₄ alkyl groups,

R9 is chosen from a hydrogen atom and C₁-C₄ linear and branched hydrocarbon chains, and

n is equal to 0 or 1.

As explained above, the process according to the present disclosure involves a fixation subsequent to the treatment step. The fixation (or oxidation) can be performed either by placing the hair in contact with the oxygen of the air or by application of an oxidizing composition comprising at least one oxidizing agent. The at least one oxidizing agent can be chosen from hydrogen peroxide and alkali bromates, and in one embodiment of the present disclosure, is hydrogen peroxide.

The treatment and the fixation can be separated by an exposure time: the composition comprising the at least one derivative of formula (1) and/or the water-soluble salts thereof can be allowed to act for a period of time ranging from 10 minutes to 3 hours, at a temperature ranging, for example, from 20° C. to 70° C.

Thus, the application of a composition comprising at least one derivatives of formula (1) and/or the water-soluble salts thereof and the subsequent fixation leads to a permanent waving of the hair.

As indicated above, the process according to the present disclosure can improve the compatibility of the treatment for waving the hair obtained with a subsequent cosmetic treatment, other than a treatment for waving the hair. For instance, the process according to the present disclosure can be beneficial when the subsequent cosmetic treatment is a coloration by oxidation.

Conventional treatments for permanent waving of the hair, such as those using thioglycolic acid and hydrogen peroxide at alkaline pH, are known to be capable of causing a degradation of the hair fiber. One consequence of this degradation is, for example, an increase in the uptake of the color during the application of an oxidative coloration to hair that has been subjected to the permanent treatment: the color obtained can be much more intense on hair previously subjected to permanent treatment than on natural hair. The difference in the uptake of the color in natural hair and in hair subjected to permanent treatment can then produce an unaesthetic effect on the appearance of the head.

Thus, when the hair is waved by using the process of the present disclosure, i.e. by treating the hair by applying to the hair a composition of neutral pH comprising at least one compound chosen from derivatives of formula (1) and the water-soluble salts thereof, and by following with fixation, the uptake of the color is limited during a subsequent coloration by oxidation and thus the divergence between the uptake of the color in hair subjected to permanent treatment and the uptake of the color in natural hair is reduced.

The coloration of hair by oxidation can be performed with the aid of at least one oxidation dye. The oxidation dyes are compounds or mixtures of compounds which, in the presence of an oxidant, for example the oxygen of air or hydrogen peroxide, are oxidized to give a colored compound or a mixture of colored compounds. The at least one oxidation dye can be selected from oxidation bases, ortho-diphenols, and coupling agents.

Oxidation bases, of the para or ortho type, are compounds that are not themselves dyes but which form a dye by a process of oxidative condensation, either with themselves or in the presence of a coupling agent or modifier. They possess functional groups, either two amino groups or one amino group and one hydroxyl group in the para or ortho position to each other.

The nature of the at least one oxidation base is not critical to the present disclosure. It may be chosen from, for example, ortho- and para-phenylenediamines, double bases, ortho- and para-aminophenols, heterocyclic bases, and the acid addition thereof.

The ortho-diphenols are compounds comprising at least one aromatic ring on which at least two consecutive carbons bear a hydroxyl group. For example, the aromatic ring can be chosen from benzene rings and condensed aromatic rings. The aromatic ring may be a condensed aromatic ring optionally comprising at least one heteroatom such as naphthalene, tetrahydronaphthalene, indane, indene, anthracene, phenanthene, indole, isoindole, indoline, isoindoline, benzofuran, dihydrobenzofuran, chroman, isochroman, chromene, isochromene, quinoline, tetrahydroquinoline and isoquinoline.

Another class of oxidation dyes is the coupling agents, as previously described above. The coupling agents may be selected from those conventionally used in oxidative dyeing, for instance, meta-aminophenols, meta-phenylenediamines, meta-diphenols, naphthols, heterocyclic coupling agents such as, for example, indole derivatives, indoline derivatives, sesamol and its derivatives, pyridine derivatives, pyrazolotriazole derivatives, pyrazolones, indazoles, benzimidazoles, benzothiazoles, benzoxazoles, 1,3-benzodioxoles, quinolines, and the acid addition salts thereof, these compounds being different from the ortho-dihydroxylated compounds described previously.

Among the coupling agents that may be used disclosed herein, non-limiting mention may be made of 2,4-diamino 1-(β-hydroxyethyloxy) benzene, 2-methyl 5-amino phenol, 5-N-(β-hydroxyethyl)amino 2-methyl phenol, 3-amino phenol, 1,3-dihydroxybenzene, 1,3-dihydroxy 2-methyl benzene, 4-chloro 1,3-dihydroxybenzene, 2-amino 4-(β-hydroxyethylamino) 1-methoxy benzene, 1,3-diamino benzene, 1,3-bis-(2,4-diaminophenoxy) propane, sesamol, 1-amino 2-methoxy 4,5-methylenedioxy benzene, α-naphthol, 6-hydroxy indole, 4-hydroxy indole, 4-hydroxy N-methyl indole, 6-hydroxyindoline, 2,6-dihydroxy 4-methyl pyridine, 1-H 3-methyl pyrazol-5-one, 1 -phenyl 3-methyl pyrazol-5-one, 2-amino 3-hydroxypyridine, 3,6-dimethyl pyrazolo [3,2-c] 1,2,4-triazole, 2,6-dimethyl-pyrazolo-[1,5-b] 1,2,4-triazole, and the acid addition salts thereof.

The at least one oxidation dye is present in the coloring composition in an amount sufficient to obtain a visible coloration. This quantity may vary to a great extent as a function of the nature of the coloring dye and the intensity of the coloration desired. For example, a suitable coloration can be obtained when the active material of the at least one oxidation dye is present in an amount such that the active coloring material content in the final coloration composition is at least 0.1 micromole, such as at least 1 micromole, per millilitre of the final composition. For example, the at least one oxidation dye in the coloration composition can be present in an amount ranging from 1 mM to 10 mM per litre, such as 5 mM per litre.

By varying the nature of the different coloring materials obtained upon oxidation, and their proportions in the composition, it is possible to vary the color of the final coloration composition. In this way a range of colors is obtained.

In addition to the at least one oxidation dye, the hair coloration compositions may comprise at least one direct dye, in order, for instance, to modify the tints by enriching them with reflections. The at least one direct dye may be chosen from, for example, neutral, cationic and anionic nitro, azo and anthraquinone dyes, and may be present in the composition in an amount ranging from 0.001% to 20% by weight, such as from 0.01% to 10% by weight, relative to the total weight of the composition.

The characteristics described in the text that follows apply to the two processes forming one aspect of the present disclosure, i.e. the process for strengthening the hair and to the process for improving the compatibility of a treatment for waving the hair with a different subsequent cosmetic treatment.

As described herein, the processes according to the present disclosure uses derivatives of formula (1) and/or water-soluble salts of these derivatives. For example, the water-soluble salts of the derivatives of formula (1) may be chosen from chlorides, bromides, fluorides and iodides. For instance, the derivatives of formula (1) and the water-soluble salts thereof can be selected from the following compounds:

:-dihydro-thiophen-2-ylideneamine hydrochloride (2-iminothiolane hydrochloride):

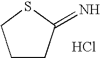

5-methyl-dihydro-thiophen-2-ylideneamine hydrochloride:

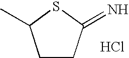

5-phenyl-dihydro-thiophen-2-ylideneamine hydrochloride:

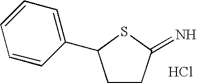

5-ter-butyl-dihydro-thiophen-2-ylideneamine hydrochloride:

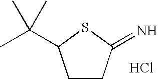

5,5-dimethyl-dihydro-thiophen-2-ylideneamine hydrochloride: 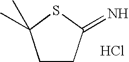
and

tetrahydro-2H-thiopyran-2-imine hydrochloride:

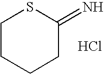

The at least one derivative of formula (1) and/or the water-soluble salts thereof can be present in an amount raging from 0.1% to 5%, such as from 0.5% to 2% by weight, relative to the total weight of the composition.

The at least one derivative of formula (1) and/or the water-soluble salts thereof are present in a cosmetically acceptable medium. This medium can comprise, for example, at least one solvent conventionally used in cosmetic compositions. Non-limiting mention may be made of, for instance, water, C₁-C₆ alcohols, benzyl alcohol, C₂-C₆ ethers, and C₂-C₆ esters. The alcohols can be chosen from, for example, alkanols, alkanediols and benzyl alcohol. Among the alkanols that may be used, non-limiting mention may be made of ethanol, propanol and isopropanol. Among the alkanediols that may be used, non-limiting mention may be made of ethylene glycol, propylene glycol, and pentanediol.

In one aspect of the present disclosure, for example, the solvent is a water/alcohol mixture.

The composition used in the processes according to the present disclosure and comprising the at least one derivative of formula (1) and/or the water-soluble salts thereof, must have a neutral pH. As used herein, the term “neutral pH” is understood to mean a pH ranging from 6 to 8, such as from 6.5 to 7.5, for instance from 7 to 7.5. For that purpose, the pH can be stabilized with the aid of a buffer solution.

As used herein, the term “buffer solution” is understood to mean a solution, the pH of which does not vary upon dilution or by the addition of a small quantity of acid or base. The formulation of buffer solutions is well-known to the person skilled in the art. In one aspect of the processes according to the present disclosure, a phosphate buffer solution is used, for example a buffer solution based on monopotassium phosphate KH₂PO₄.

The composition according to the present disclosure may also comprise at least one cosmetically acceptable adjuvant conventionally used in the cosmetic compositions intended for application to the hair and which are not prejudicial to the properties desired. Among the adjuvants that may be used, non-limiting mention may be made, for example, of gelling agents and/or thickeners, anionic, non-ionic, cationic or amphoteric surfactants, propenetrant agents, emulsifiers, perfumes, preservatives, fillers, sun filters, coloring materials, proteins, vitamins, provitamins, anionic, non-ionic, cationic or amphoteric non-fixing polymers, hydrating agents, emollients, softening agents, mineral, vegetable or synthetic oils, hydrophilic or lipophilic active agents like the ceramides and the pseudoceramides, anti-foam agents, antiperspirant agents, anti-free radical agents, fixing polymers, bactericidal agents and anti-pellicular agents.

The composition used in the processes according to the present disclosure may be available in the form of a lotion, thickened or not, a cream thickened or not, a gel or any other appropriate form for treating hair. For instance, optionally, it can be packaged in a spray bottle or in an aerosol container.

In the processes according to the present disclosure, the composition comprising at least one derivative of formula (1) is applied to the hair for a period of time sufficient to obtain the desired effect. For example, the period of time may range from 10 minutes to 3 hours. For instance, the composition can be applied for a period of time ranging from 1.5 hours to 2.5 hours.

Similarly, in the processes according to the present disclosure, the composition comprising at least one derivative of formula (1) can be applied to the hair at a temperature ranging from 20° C. to 70° C., such as from 40° C. to 60° C.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific example are reported as precisely as possible. Any numerical value, however, inherently contain certain errors necessarily resulting from the standard deviation found in their respective testing measurements.

The following examples are intended to illustrate the invention in a non-limiting. manner.

### EXAMPLES

### Example 1

The efficacy of the waving obtained by a treatment process according to the present disclosure is measured.
(a) INVENTIVE: A solution comprising 1% by weight of 2-iminothiolane hydrochloride (sold by the ALDRICH CHEM CO under the catalogue number 33,056-6) was prepared in phosphate buffer. The composition of the phosphate buffer was the following:
0.05M phosphate buffer (6.8 g of KH₂PO₄-PROLABO NORMA PUR)
water qsp 200 ml
2N KOH qsp pH=7.84.

A lock of 90% white natural hair was wrapped around a curler and introduced into this solution, the solution being in a quantity such that the bath ratio was 10 ml of solution for 1 gram of hair. The final pH of the solution was 7.4.

The bath was left to stand at 50° C. for 2 hours.

After rinsing, fixation was performed on the hair previously treated: a solution of 8 volumes hydrogen peroxide at pH 3 (obtained by the introduction of citric acid) was applied for 10 minutes at 30° C. The bath ratio was 10 ml of oxidizing solution for 1 gram of hair.
(b) COMPARATIVE: A comparative treatment for permanent waving of the hair

according to the prior art was performed on 90% white hair derived from the same batch used in (a).

A lock of hair was rolled on a curler and subjected to reduction by an aqueous solution comprising 1 mol/L thioglycolic acid and ammonium hydroxide in an amount sufficient to obtain an alkaline pH of 8.5. The bath ratio was 10 ml of solution for 1 gram of hair.

The bath was left to stand at 30° C. for 15 minutes.

After rinsing, oxidation was performed on the hair: a solution of 8 volumes of hydrogen peroxide at pH 3 (obtained by the introduction of citric acid) was applied for 10 minutes at 30° C. The bath ratio was 10 ml of oxidizing solution for 1 gram of hair.
(c) CONTROL: A lock of 90% white hair derived from the same batch as those previously treated was rolled on a curler, but not subjected to any treatment. This lock served as control.

The 3 curled locks previously prepared were rinsed, dried and then introduced into a glove box at controlled relative humidity (45%) and controlled temperature (25° C.) for 12 hours. The curlers were withdrawn from the locks which were then placed in a glove box at controlled relative humidity (80%) and controlled temperature (25° C.) for 1 hour. The length of the loop (defined by the diameter of the curled portion, in millimetres) measured at (time=t) t=0, t=30 minutes and t=1 hour made it possible to quantify the relaxation of the hair and hence to characterise the durability of the treatment applied. The results are presented in Table 1.

TABLE 1

| | | Hair treated | Hair treated with 2- |
|---|---|---|---|
| | Control | with thioglycolic | iminothiolane HCl |
| | hair | acid pH 8.5 | pH 7.4 |
| Length of the | 8.5 | 7 | 7.5 |
| lock initially | | | |
| (mm) | | | |
| Length of the | 15.5 | 9.5 | 12.5 |
| lock at | | | |
| t = 30 min (mm) | | | |
| Length of the | 16.5 | 12.5 | 14 |
| lock at t = 1 h | | | |
| (mm) | | | |

These results show that the treatment with 2-iminothiolane hydrochloride at neutral pH leads to permanent waving.

### Example 2

In this example, the compatibility of the waving carried out by the treatment process according to the present disclosure with a subsequent coloration by oxidation was assessed.

A coloration by oxidation was carried out on the 3 locks of hair prepared according to Example 1, i.e. the lock treated with 2-iminothiolane hydrochloride, the lock treated with thioglycolic acid and the control lock.

The coloration was carried out by using the commercial formula of L'OREAL Professionnel Crescendo 7.44 according to the protocol described in the directions for use of the product.

The mixture was applied to the hair in a bath ratio of 10 g of composition for 1 g of hair. The locks were treated for 30 minutes at 30° C. then rinsed, washed with a 2% shampoo solution Dop® Camomille in water, and dried at room temperature.

The color of the locks was then measured with the aid of a spectrocolorimeter Minolta CM2022 in order to assess the uptake of the color in each case. The measurements were made in the color space L*a*b.

The uptake of the color ΔE was quantified by the value of the color deviation ΔE*ab defined by the following equation:
ΔE*ab=[(ΔL*)²+(Δa*)²+(Δb*)²]^{1/2}.

The results are presented in Table 2.

TABLE 2

| | | Hair treated | |
|---|---|---|---|
| | | with thioglycolic | Hair treated |
| | | acid | with 2-iminothiolane |
| | Natural hair | pH 8.5 | HCl, pH 7.0004 |
| Uptake of the | 19.8 +/− 1.0 | 29.0 +/− 0.5 | 23.9 +/− 1.4 |
| color (ΔE) | | | |

The results show that the treatment with 2-iminothiolane hydrochloride at neutral pH made it possible to diminish the uptake of the color Crescendo 7.44 and to thus reduce the divergence from the color achieved on natural hair that has not been the object of a waving treatment.

Accordingly these results show a better compatibility of a waving treatment with 2-iminothiolane hydrochloride with a subsequent coloration by oxidation, than of a treatment with thioglycolic acid with a subsequent coloration by oxidation.

### Example 3

In this example, the strengthening of the hair provided by the treatment with 2-iminothiolane was assessed.
(a) A solution comprising 1% by weight of 2-iminothiolane hydrochloride (sold by the ALDRICH CHEM CO under the catalogue number 33,056-6) was prepared in phosphate buffer. The composition of the phosphate buffer was the following:
   0.05M phosphate buffer (6.8 g of KH₂PO₄-PROLABO NORMA PUR)
   water qsp 200 ml p2 2N KOH qsp pH=7.84.

A lock of 90% white natural hair was introduced into this solution, the solution being in an amount such that the bath ratio was 10 ml of solution for 1 gram of hair. The final pH of the solution was 7.4.

It was left to stand at 50° C. for 2 hours.

After rinsing, fixation was performed on the hair previously treated: a solution of 8 volumes hydrogen peroxide at pH 3 (obtained by the introduction of citric acid) was applied for 10 minutes at 30° C. The bath ratio was 10 ml of oxidizing solution for 1 gram of hair.
(b) A lock of 90% white hair derived from the same batch as previously used underwent no treatment. This lock served as control.

The 2 locks (a) and (b) were rinsed, dried, then introduced into a glove box at controlled relative humidity (45%) and controlled temperature (25° C.) for 12 hours.

A panel of experts judged the hair treated with 2-iminothiolane strengthened compared with untreated hair.

1. A process for strengthening hair, comprising treating the hair by applying to the hair a composition of neutral pH, comprising, in a cosmetically acceptable medium, at least one compound chosen from derivatives of 2-iminothiolane of formula (1), and the water-soluble salts thereof: 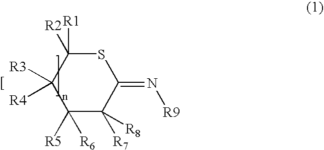
wherein:
R1 to R8, which may be identical or different, are chosen from hydrogen atoms;
linear and branched C₁-C₄ hydrocarbon chains, optionally comprising at least one unsaturation, optionally substituted with at least one group chosen from thiol, C₁-C₄ thioalkyl, C₁-C₄ alkylthiol, C₂-C₈ N,N-dialkylamine, C₂-C₄ alkylcarbonylthiol and C₂-C₄ N-alkylcarbonylamine groups; and C₆ aryl groups optionally substituted with one entity chosen from halogen atoms, thiol groups, and linear and branched C₁-C₄ alkyl groups,
R9 is chosen from a hydrogen atom and C₁-C₄ linear and branched hydrocarbon chains, and
n is equal to 0 or 1;
wherein the at least one compound is present in an amount effective to strengthen the hair.

2. The process according to claim 1, wherein said process is performed independently of any other hair treatment.

3. The process according to claim 1, wherein said process is performed in combination with a second hair treatment, either before, during or after said treatment.

4. The process according to claim 3, wherein the second hair treatment is chosen from a shampoo, an after-shampoo, a permanent or semi-permanent waving, a straightening, and a hairdressing.

5. A process for treating the hair, comprising
applying to the hair a waving composition at neutral pH, and
fixing the treated hair,
wherein the waving composition comprises, in a cosmetically acceptable medium, at least one compound chosen from derivatives of 2-iminothiolane of formula (1) and the water-soluble salts thereof: 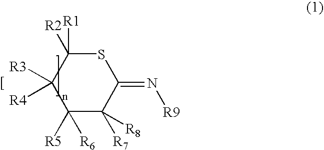
wherein
R1 to R8, which may be identical or different, are chosen from hydrogen atoms;
linear and branched C₁-C₄ hydrocarbon chains, optionally comprising at least one unsaturation, optionally substituted with at least one group chosen from thiol, C₁-C₄ thioalkyl, C₁-C₄ alkylthiol, C₂-C₈ N,N-dialkylamine, C₂-C₄ alkylcarbonylthiol and C₂-C₄ N-alkylcarbonylamine groups; and C₆ aryl groups optionally substituted with at least one entity chosen from halogen atoms, thiol groups and linear and branched C₁-C₄ alkyl groups,
R9 is chosen from a hydrogen atom and C₁-C₄ linear and branched hydrocarbon chains,
n is equal to 0 or 1; and
wherein the waving composition comprises the at least one compound in an amount effective to allow compatibility of the said waving process hair with a subsequent different cosmetic treatment process, wherein said subsequent different cosmetic treatment process is not a waving process.

6. The process according to claim 5, wherein the fixation of the hair comprises placing the hair in contact with the oxygen of the air, or applying to the hair an oxidizing composition comprising at least one oxidizing agent.

7. The process according to claim 6, wherein the at least one oxidizing agent is chosen from hydrogen peroxide and alkaline bromates.

8. The process according to claim 7, wherein the at least one oxidizing agent is hydrogen peroxide.

9. The process according to claim 5, wherein the subsequent different cosmetic treatment is a coloration by oxidation.

10. The process according to claim 9, wherein the coloration by oxidation comprises applying to the hair a composition comprising at least one oxidation dye chosen from oxidation bases, ortho-diphenols, and coupling agents.

11. The process according to claim 1, wherein the water-soluble salts of the derivatives of formula (1) are chosen from chlorides, bromides, fluorides and iodides.

12. The process according to claim 5, wherein the water-soluble salts of the derivatives of formula (1) are chosen from chlorides, bromides, fluorides and iodides.

13. The process according to claim 1, wherein the at least one compound is chosen from:
dihydro-thiophen-2-ylideneamine hydrochloride (2-iminothiolane hydrochloride): 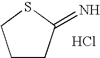
5-methyl-dihydro-thiophen-2-ylideneamine hydrochloride: 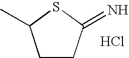
5-phenyl-dihydro-thiophen-2-ylideneamine hydrochloride: 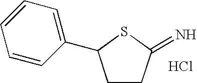
5-ter-butyl-dihydro-thiophen-2-ylideneamine hydrochloride: 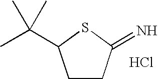
5,5-dimethyl-dihydro-thiophen-2-ylideneamine hydrochloride: 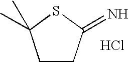
tetrahydro-2H-thiopyran-2-imine hydrochloride:

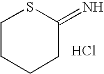

14. The process according to claim 5, wherein the at least one compound is chosen from:
dihydro-thiophen-2-ylideneamine hydrochloride (2-iminothiolane hydrochloride): 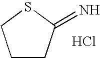
5-methyl-dihydro-thiophen-2-ylideneamine hydrochloride: 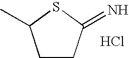
5-phenyl-dihydro-thiophen-2-ylideneamine hydrochloride: 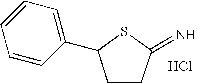
5-ter-butyl-dihydro-thiophen-2-ylideneamine hydrochloride: 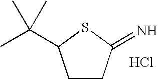
5,5-dimethyl-dihydro-thiophen-2-ylideneamine hydrochloride: 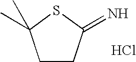
tetrahydro-2H-thiopyran-2-imine hydrochloride:

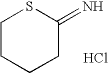

15. The process according to claim 1, wherein the at least one compound is present in an amount ranging from 0.1% to 5%, by weight, relative to the total weight of the composition.

16. The process according to claim 15, wherein the at least one compound is present in an amount ranging from 0.5% to 2%, by weight, relative to the total weight of the composition.

17. The process according to claim 5, wherein the at least one compound is present in an amount ranging from 0.1% to 5%, by weight, relative to the total weight of the composition.

18. The process according to claim 17, wherein the at least one compound is present in an amount ranging from 0.5% to 2%, by weight, relative to the total weight of the composition.

19. The process according to claim 1, wherein the composition comprises at least one solvent chosen from water, C₁-C₆ alcohols, benzyl alcohol, C₂-C₆ ethers, and C₂-C₆ esters.

20. The process according to claim 19, wherein the at least one solvent is choen from alkanols, alkanediols, and benzyl alcohol.

21. The process according to claim 20, wherein the alkanols are chosen from ethanol, propanol, and isopropanol, and/or the alkanediols are chosen from ethylene glycol, propylene glycol, and pentanediol.

22. The process according to claim 5, wherein the composition comprises at least one solvent chosen from water, C₁-C₆ alcohols, benzyl alcohol, C₂-C₆ ethers, and C₂-C₆ esters.

23. The process according to claim 22, wherein the at least one solvent is choen from alkanols, alkanediols, and benzyl alcohol.

24. The process according to claim 23, wherein the alkanols are chosen from ethanol, propanol, and isopropanol, and/or the alkanediols are chosen from ethylene glycol, propylene glycol, and pentanediol.

25. The process according to claim 19, wherein the at least one solvent is a water/alcohol mixture.

26. The process according to claim 22, wherein the at least one solvent is a water/alcohol mixture.

27. The process according to claim 1, wherein the pH of the composition ranges from 6.5 to 7.5.

28. The process according to claim 5, wherein the pH of the composition ranges from 6.5 to 7.5.

29. The process according to claim 27, wherein the pH of the composition is stabilized by a buffer solution.

30. The process according to claim 29, wherein the buffer solution is a phosphate buffer solution.

31. The process according to claim 28, wherein the pH of the composition is stabilized by a buffer solution.

32. The process according to claim 31, wherein the buffer solution is a phosphate buffer solution.

33. The process according to claim 1, wherein the composition further comprises at least one cosmetic adjuvant chosen from gellants and/or thickeners; anionic, non-ionic, cationic and amphoteric surfactants; propenetrant agents; emulsifiers; perfumes; preservatives; fillers; sun filters; coloring materials; proteins; vitamins; provitamins, anionic, non-ionic, cationic and amphoteric non-fixing polymers; hydrating agents; emollients; softening agents; mineral, vegetable and synthetic oils; hydrophilic and lipophilic active agents; anti-foam agents; antiperspirant agents; anti-free radical agents; fixing polymers; bactericidal agents; and anti-pellicular agents.

34. The process according to claim 5, wherein the composition further comprises at least one cosmetic adjuvant chosen from gellants and/or thickeners; anionic, non-ionic, cationic and amphoteric surfactants; propenetrant agents; emulsifiers; perfumes; preservatives; fillers; sun filters; coloring materials; proteins; vitamins; provitamins, anionic, non-ionic, cationic and amphoteric non-fixing polymers; hydrating agents; emollients; softening agents; mineral, vegetable and synthetic oils; hydrophilic and lipophilic active agents; anti-foam agents; antiperspirant agents; anti-free radical agents; fixing polymers; bactericidal agents; and anti-pellicular agents.

35. The process according to claim 1, wherein the composition is in a form chosen from a lotion, a cream or a gel.

36. The process according to claim 5, wherein the composition is in a form chosen from a lotion, a cream or a gel.

37. The process according to claim 1, wherein the composition is applied to the hair for a period of time ranging from 10 minutes to 3 hours.

38. The process according to claim 37, wherein the composition is applied to the hair for a period of time ranging from 1.5 hours to 2.5 hours.

39. The process according to claim 5, wherein the composition is applied to the hair for a period of time ranging from 10 minutes to 3 hours.

40. The process according to claim 39, wherein the composition is applied to the hair for a period of time ranging from 1.5 hours to 2.5 hours.

41. The process according to claim 1, wherein the composition is applied to the hair at a temperature ranging from 20° C. to 70° C.

42. The process according to claim 41, wherein the composition is applied to the hair at a temperature ranging from 40° C. to 60° C.

43. The process according to claim 5, wherein the composition is applied to the hair at a temperature ranging from 20° C. to 70° C.

44. The process according to claim 43, wherein the composition is applied to the hair at a temperature ranging from 40° C. to 60° C.
